(19) United States
(12) Patent Application Publication
(10) Pub. No.: US 2009/0308396 A1
(43) Pub. Date: Dec. 17, 2009

(54) Wearable Oxygen Concentrator System

(75) Inventor: Michael S. McClain, Ortonville, MI (US)

Correspondence Address: DELPHI TECHNOLOGIES, INC., M/C 480-410-202, PO BOX 5052, TROY, MI, 48007, US

(73) Assignee: DELPHI TECHNOLOGIES, Troy, MI (US)

(21) Appl. No.: 12/138,804

(22) Filed: Jun. 13, 2008

(51) Int. Cl. A62B 7/00; A61M 15/08

(52) U.S. Cl. 128/205.11; 128/205.22; 128/207.18

(57) ABSTRACT

A wearable oxygen concentrator system is provided. The wearable oxygen concentrator system includes a pumping system configured to receive air and to remove nitrogen gas from the air to obtain concentrated oxygen. The pumping system is further configured to deliver the concentrated oxygen via a cannula tube to a person. The wearable oxygen concentrator system further includes a clothing member configured to be worn by the person. The clothing member is configured to hold the pumping system therein.

### BACKGROUND

An oxygen concentrator has been utilized to output concentrated oxygen. However, a drawback with the oxygen concentrator is that it is housed in a mobile cart that is pulled by a person receiving oxygen from the oxygen concentrator. Accordingly, a person's mobility is relatively limited due to the mobile cart.

Accordingly, the inventor herein has recognized a need for a wearable oxygen concentrator system that minimizes and/or reduces the above-mentioned deficiency.

### SUMMARY

A wearable oxygen concentrator system in accordance with an exemplary embodiment is provided. The wearable oxygen concentrator system includes a pumping system configured to receive air and to remove nitrogen gas from the air to obtain concentrated oxygen. The pumping system is further configured to deliver the concentrated oxygen via a cannula tube to a person. The wearable oxygen concentrator system further includes a clothing member configured to be worn by the person. The clothing member is configured to hold the pumping system therein.

### BRIEF DESCRIPTION OF THE DRAWINGS

FIG. 1 is a schematic of a wearable oxygen concentrator system in accordance with an exemplary embodiment;

FIG. 2 is a schematic of a pumping system utilized in the wearable oxygen concentrator system of FIG. 1;

FIG. 3 is a timing schematic illustrating operation of the pumping system of FIG. 2; and

FIG. 4 is a schematic of a wearable oxygen concentrator system in accordance with another exemplary embodiment.

### DESCRIPTION OF EXEMPLARY EMBODIMENTS

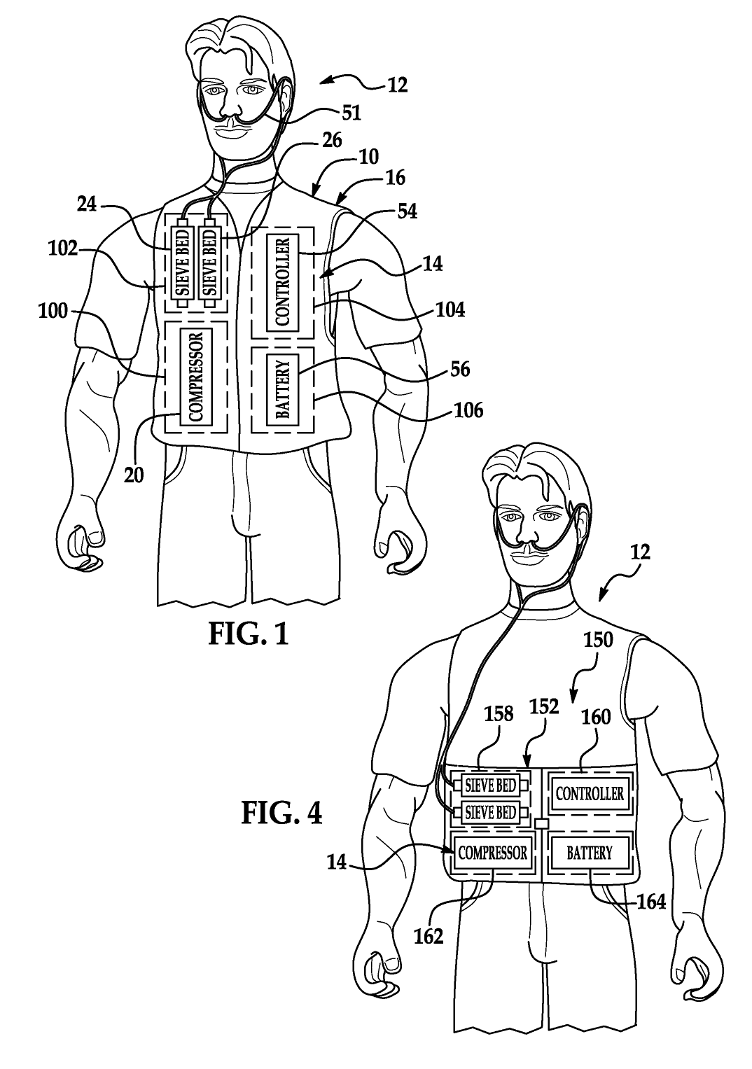

Referring to FIGS. 1 and 2, a wearable oxygen concentrator system 10 that can be worn by a person 12 in accordance with an exemplary embodiment is illustrated. The wearable oxygen concentrator 10 includes a pumping system 14, and a clothing member or article of clothing such as a jacket 16 which can be worn around a chest of the person 12.

The pumping system 14 is provided to deliver concentrated oxygen through a cannula tube 51 to the person 12. The pumping system 14 includes a compressor 20, a filter 22, sieve beds 24, 26, an inlet manifold 27, inlet valves 28, 30, vent valves 32, 34, outlet valves 36, 38, a counter-fill valve 40, an outlet manifold 41, an oxygen sensor 42, a particulate filter 44, a cannula tip 46, tubes 48, 50, a cannula tube 51, a fan 53, a controller 54, a battery 56, an input device 58, a display device 60, and the jacket 16.

The jacket 16 is configured to hold components of the pumping system 14 therein. In one exemplary embodiment, the jacket 16 has a pocket 100 for holding the compressor 20 therein. Further, the jacket 16 has a pocket 102 for holding the sieve beds 24, 26 therein. Further, the jacket 16 has a pocket 104 for holding the controller 54 therein. Further, the jacket 16 has a pocket 104 for holding the battery 56 therein. In one exemplary embodiment, the remaining components of the pumping system 14, excluding the cannula tube 51, are disposed in one or more of the pockets 100, 102, 104 and 106. Of course, in an alternative embodiment, the jacket 16 can have additional pockets therein for holding the remaining components of the pumping system 14. Further, in another alternative embodiment, the jacket 16 can have the pockets disposed in locations different from those locations shown in FIG. 1.

In one exemplary embodiment, the jacket 16 is constructed from a lightweight, durable and water resistant material. For example, the jacket 16 can be constructed from a weaved material constructed from at least one of: (i) vinyl, (ii) polyester, (iii) Gortex, (iv) nylon and (v) a combination of the foregoing materials. Further, the jacket 16 may have a foam layer attached to an inner side of the lightweight, durable and water resistant material, which would provide a thermal barrier, and sound and vibration dampening of internal components of the jacket 16. The foam layer can be constructed from at least one of: (i) a melamine foam, (ii) a polyurethane foam, (iii) a polyethylene foam, and (iv) a vinyl foam.

Referring to FIG. 2, the compressor 20 is configured to output compressed air in response to a first control signal from the controller 54. The compressor 20 is fluidly coupled to the filter 22 and the filter 22 receives the compressed air from the compressor 20. Further, the filter 22 filters the compressed air passing therethrough and routes the compressed air to the tube 23. The tube 23 routes the compressed air to the intake manifold 27 which further routes the compressed air to the inlet valves 28, 30.

The inlet valve 28 is configured to have an open operational position to route compressed air into the sieve bed 24 in response to a second control signal from the controller 54. Further, the inlet valve 28 is configured to have a closed operational position that stops routing compressed air into the sieve bed 24 when the controller 54 stops outputting the second control signal.

The inlet valve 30 is configured to have an open operational position to route compressed air into the sieve bed 26 in response to a third control signal from the controller 54. Further, the inlet valve 30 is configured to have a closed operational position that stops routing compressed air into the sieve bed 26 when the controller 54 stops outputting the third control signal.

The sieve bed 24 is configured to remove nitrogen gas from the compressed air received from the inlet valve 28. The sieve bed 24 includes an active region 80 and a collection region 82. The active region 80 comprises a nitrogen gas collecting material which removes nitrogen gas from the receive compressed air. The collection region 82 collects concentrated oxygen therein. Further, the sieve bed 24 is purged of the nitrogen gas when the inlet vent valve 28 has a closed operational position and the vent valve 32 fluidly coupled to the sieve bed 24 has an open operational position.

The sieve bed 26 is configured to remove nitrogen gas from the compressed air received from the inlet valve 30. The sieve bed 26 includes an active region 84 and a collection region 86. The active region 84 comprises a nitrogen gas collecting material which removes nitrogen gas from the receive compressed air. The collection region 86 collects concentrated oxygen therein. Further, the sieve bed 26 is purged of the nitrogen gas when the inlet vent valve 30 has a closed operational position and the vent valve 34 fluidly coupled to the sieve bed 26 has an open operational position.

The vent valve 32 is configured to have an open operational position to vent nitrogen gas from the sieve bed 24 in response to a fourth control signal from the controller 54. Further, the vent valve 32 is configured to have a closed operational position that stops venting nitrogen gas from the sieve bed 24 when the controller 54 stops outputting the fourth control signal.

The vent valve 34 is configured to have an open operational position to vent nitrogen gas from the sieve bed 26 in response to a fifth control signal from the controller 54. Further, the vent valve 34 is configured to have a closed operational position that stops venting nitrogen gas from the sieve bed 26 when the controller 54 stops outputting the fifth control signal.

The counter-fill valve 40 is configured to have an open operational position to route compressed gas between the sieve beds 24, 26 to assist in venting nitrogen gas from the sieve beds 24, 26, in response to a sixth control signal from the controller 54. Further, the counter-fill valve 40 is configured to have a closed operational position when the controller 54 stops outputting the sixth control signal.

The outlet manifold 41 is fluidly coupled to the sieve beds 24, 26 and to the outlet valves 36, 38. The outlet manifold 41 is configured to route concentrated oxygen from the sieve beds 24, 26 to the outlet valves 36, 38 respectively.

The outlet valve 36 is configured to have an open operational position to deliver concentrated oxygen from the sieve bed 24 to the tube 48 in response to a seventh control signal from the controller 54. Further, the outlet valve 36 is configured to have a closed operational position to stop delivering oxygen from the sieve bed 24 to the tube 48 when the controller 54 stops outputting the seventh control signal.

The outlet valve 38 is configured to have an open operational position to deliver concentrated oxygen from the sieve bed 26 to the tube 48 in response to an eighth control signal from the controller 54. Further, the outlet valve 38 is configured to have a closed operational position to stop delivering oxygen from the sieve bed 26 to the tube 48 when the controller 54 stops outputting the eighth control signal.

The outlet valves 36, 38 fluidly communicate with the cannula tip 46 via a flow path defined by the tube 48, the oxygen sensor 42, the tube 50, and the filter 44. In particular, the outlet valves 36, 38 deliver concentrated oxygen to the cannula tip 46 that is routed through the cannula tube 51 to the person 12.

The fan 53 is configured to blow air toward the compressor 20 for cooling the compressor 20 in response to a ninth control signal from the controller 54. The battery 56 is configured to generate an operational voltage that is received by the controller 54. The display device 60 is configured to display information indicating the amount of concentrated oxygen being delivered to the person 12. The input device 58 is configured to allow the person 12 to input data that adjusts an amount of concentrated oxygen delivered to the person 12.

The controller 54 is configured to generate control signals for controlling operation of the compressor 20, the inlet valves 28, 30, the vent valves 32, 34, the outlet valves 36, 38, the counter-fill valve 40, the fan 53, and the display device 60. Further, the controller 54 is configured to receive data from the input device 58 and an oxygen concentration signal from the oxygen sensor 42.

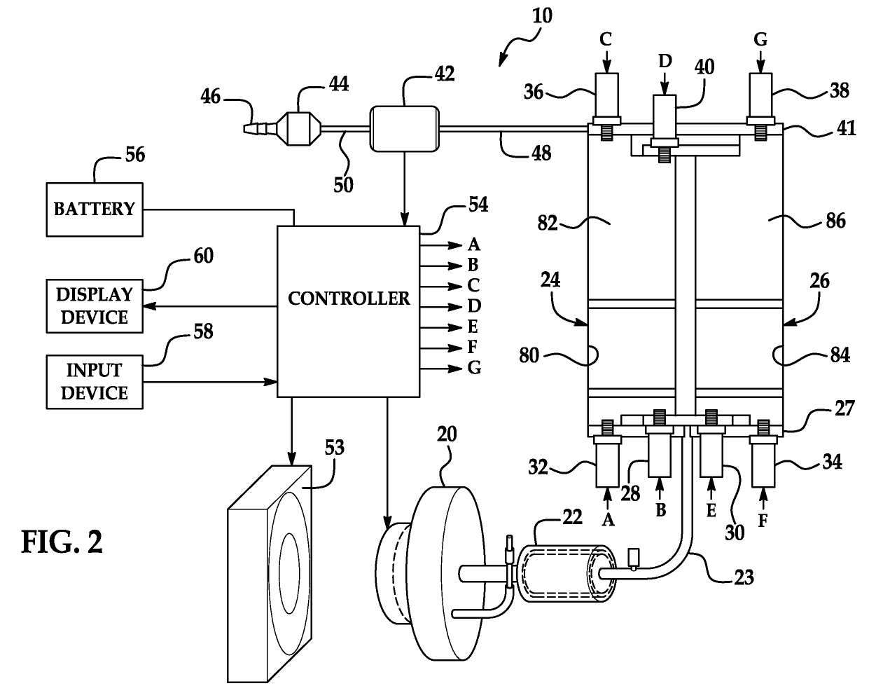

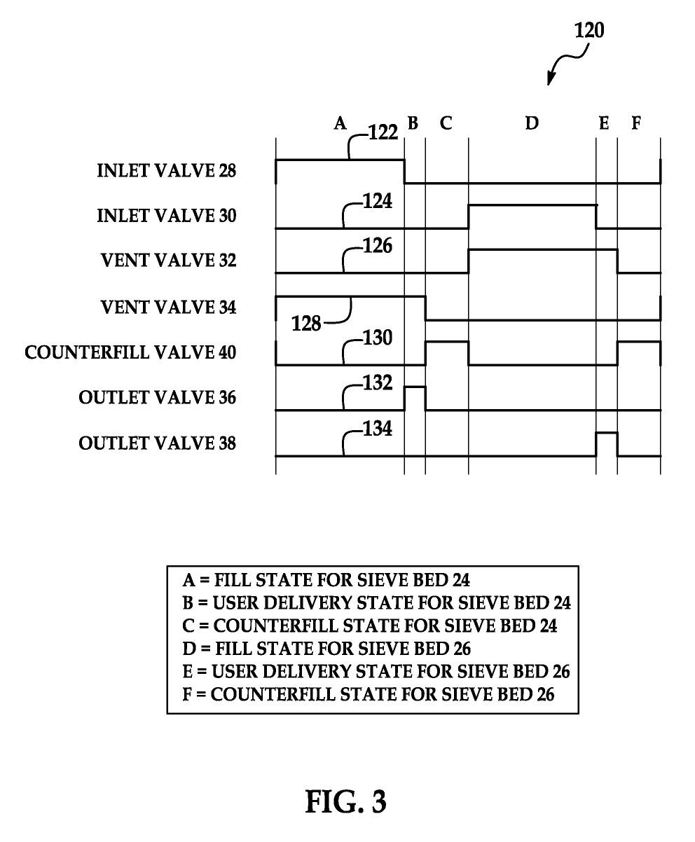

Referring to FIG. 3, in one exemplary embodiment, the controller 54 controls the pumping system 14 in accordance with the timing schematic 120. The timing schematic 120 includes timing curves 122, 124, 126, 128, 130, 132, 134. In particular, during time interval “A”, the sieve bed 24 is being filled with compressed air via the inlet valve 28 as shown by timing curve 122. During time interval “B”, the person 12 is receiving concentrated oxygen from the sieve bed 24 as shown by timing curve 132. During time interval C, the sieve bed 24 is receiving compressed gas from the sieve bed 26 via the counter-fill valve 40, to vent nitrogen gas from the sieve bed 24, as shown by timing curve 130. During time interval “D”, the sieve bed 26 is being filled with compressed air via the inlet valve 30 as shown by timing curve 124. During time interval “E”, the person 12 is receiving concentrated oxygen from the sieve bed 24 as shown by timing curve 134. During time interval “F”, the sieve bed 26 is receiving compressed gas from the sieve bed 24 via the counter-fill valve 40, to vent nitrogen gas from the sieve bed 26, as shown by timing curve 130.

Referring to FIG. 4, a wearable oxygen concentrator system 150 that can be worn by a person 12 in accordance with another exemplary embodiment is illustrated. The wearable oxygen concentrator 150 includes the pumping system 14, and a clothing member such as a belt 152. As shown, the belt 152 can be disposed around a waist of the person 12.

The belt 152 is configured to hold components of the pumping system 14 therein. In one exemplary embodiment, the belt 152 has a pocket 162 for holding the compressor 20 therein. Further, the belt 152 has a pocket 158 for holding the sieve beds 24, 26 therein. Further, the belt 152 has a pocket 160 for holding the controller 54 therein. Further, the belt 152 has a pocket 164 for holding the battery 56 therein. In one exemplary embodiment, the remaining components of the pumping system 14, excluding the cannula tube 51, are disposed in one or more of the pockets 158, 160, 162 and 164. Of course, in an alternative embodiment, the belt 152 can have additional pockets therein for holding the remaining components of the pumping system 14. Further, in another alternative embodiment, the belt 152 can have the pockets disposed in locations different from those locations shown in FIG. 4.

In one exemplary embodiment, the belt 152 is constructed from a lightweight, durable and water resistant material. For example, the belt 152 can be constructed from a weaved material constructed from at least one of: (i) vinyl, (ii) polyester, (iii) Gortex, (iv) nylon and (v) a combination of the foregoing materials. Further, the belt 152 may have a foam layer attached to an inner side of the lightweight, durable and water resistant material, which would provide a thermal barrier, and sound and vibration dampening of internal components of the belt 152. The foam layer can be constructed from at least one of: (i) a melamine foam, (ii) a polyurethane foam, (iii) a polyethylene foam, and (iv) a vinyl foam.

The wearable oxygen concentrator systems provide a substantial advantage of other systems. In particular, the wearable oxygen concentrator systems have an article of clothing worn by a person that houses the components of the pumping system to allow the person to have greater mobility as compared with other systems.

While the invention has been described with reference to exemplary embodiments, various changes may be made and equivalents may be substituted for elements thereof without departing from the scope of the invention. In addition, many modifications may be made to adapt a particular situation or material to the teachings of the invention without departing from the essential scope thereof Therefore, it is intended that the invention not be limited to the particular embodiments disclosed herein, but that the invention will include all embodiments falling within the scope of the appended claims.

What is claimed is:

1. A wearable oxygen concentrator system, comprising:
a pumping system configured to receive air and to remove nitrogen gas from the air to obtain concentrated oxygen, the pumping system further configured to deliver the concentrated oxygen via a cannula tube to a person; and
a clothing member configured to be worn by the person, the clothing member configured to hold the pumping system therein.

2. The wearable oxygen concentrator system of claim 1, wherein the pumping system includes:
a compressor configured to receive the air and to output compressed air in response to a first control signal;
first and second inlet valves configured to receive the compressed air from the compressor;
first and second sieve beds fluidly coupled to the first and second inlet valves, respectively, the first and second inlet valves configured to deliver compressed air to the first and second sieve beds, respectively, in response to second and third control signals, respectively, the first and second sieve beds configured to remove the nitrogen gas from the compressed air to obtain concentrated oxygen;
first and second outlet valves fluidly coupled to the first and second sieve beds configured to receive the concentrated oxygen from the first and second sieve beds; and
a particulate filter fluidly coupled to the first and second outlet valves, the first and second outlet valves configured to deliver concentrated oxygen to the particulate filter in response to fourth and fifth control signals, respectively, the particulate filter configured to remove particulates from the concentrated oxygen, the particulate filter being fluidly to the cannula tube.

3. The wearable oxygen concentrator system of claim 2, wherein the clothing member has a pocket for holding the compressor therein.

4. The wearable oxygen concentrator system of claim 2, wherein the clothing member has a pocket for holding the first and second sieve beds therein.

5. The wearable oxygen concentrator system of claim 2, further comprising a battery electrically coupled to the compressor.

6. The wearable oxygen concentrator system of claim 5, wherein the clothing member has a pocket for holding the battery therein.

7. The wearable oxygen concentrator system of claim 2, further comprising a controller configured to generate the first, second, third, fourth, and fifth control signals.

8. The wearable oxygen concentrator system of claim 7, wherein the clothing member has a pocket for holding the controller therein.

9. The wearable oxygen concentrator system of claim 1, wherein the clothing member comprises a jacket configured to be worn on the chest of the person.

10. The wearable oxygen concentrator system of claim 1, wherein the clothing member comprises a belt configured to be worn about a waist of the person.
